(19) United States
(12) Patent Application Publication
(10) Pub. No.: US 2025/0134676 A1
(43) Pub. Date: May 1, 2025

(54) IMPLANT INSERTION APPARATUS AND METHOD OF USE

(71) Applicant: Pressio, Inc., Memphis, TN (US)

(72) Inventors: David J. Pancratz, Helotes, TX (US); Natalie Leticia Miguel, San Antonio, TX (US); John L. White, Arlington, TX (US); James P. Duncan, Hernando, MS (US)

(21) Appl. No.: 18/932,532

(22) Filed: Oct. 30, 2024

Related U.S. Application Data
(60) Provisional application No. 63/594,256, filed on Oct. 30, 2023.

(51) Int. Cl. A61F 2/46

(52) U.S. Cl. CPC A61F 2/4603; A61F 2002/4627

(57) ABSTRACT

The present disclosure relates to various embodiments of an inserter assembly system and methods of using the same. The inserter assembly includes a housing comprising an inner surface extending within the housing, the inner surface comprising a recess, and an opening designed to receive a portion of a knob. The inserter assembly includes a bender fork received within and selectively engaging with the recess, the bender fork comprising a threaded opening and a prong, the prong further comprising a hook. The inserter assembly includes a push rod comprising a cavity sized to receive a locking pin, wherein the locking pin comprises locking pin threads. The knob is designed to rotate within the opening and selectively engage with the locking pin. The locking pin threads selectively engage with the threaded opening. The bender fork translates within the recess between a first bender fork position and a second bender fork position.

### CROSS-REFERENCE TO RELATED APPLICATIONS

This application claims the benefit of priority to U.S. Provisional Patent Application No. 63/594,256 filed Oct. 30, 2023, which is incorporated herein by reference in its entirety.

### BACKGROUND

Implants such as orthopedic staples or other fixation-type implants are devices used in surgical procedures to help stabilize patient anatomy (e.g., bones, soft tissues) and support load during the healing process. For instance, during a surgical procedure, corrective cuts may be made to underlying patient anatomy to prepare the patient anatomy for an implant to be inserted into or around the cuts. While fixation-type implants can provide a simple approach to ensure patient healing, the transportation (e.g., storage, delivery, insertion, retrieval) of these implants can be difficult due to a number of factors that may include their stored energy and force. Over time, certain mechanical components of the implants can bind, malfunction, crack/warp, or cold weld, which can impede transportation of the implant to or from the patient anatomy.

As a result, there is a need for improved implant insertion and retrieval devices and methods of producing and using the same.

### BRIEF SUMMARY OF THE DISCLOSURE

Briefly described, aspects of the present disclosure generally relate to an inserter assembly system designed to insert or receive a surgical implant, as well as processes for using the same.

According to a first aspect, the present disclosure relates to a system for inserting or retrieving a surgical implant, the system comprising: a housing having a first end and a second end, the housing defining a hollow interior compartment, the housing comprising: an inner surface extending internally within the housing between the first end and the second end of the housing, the inner surface comprising a recess; and an opening disposed at the first end of the housing, the opening designed to receive a portion of a knob; a bender fork having a first end and a second end, the bender fork received within and selectively engaging with the recess, the bender fork comprising: a threaded opening disposed at the first end of the bender fork; and a prong, the prong further comprising a hook disposed at the second end of the bender fork; a push rod having a first end and a second end, the push rod positioned adjacent to the bender fork, push rod comprising: a cavity positioned at the first end of the push rod, the cavity sized to receive a second end of a locking pin; wherein the locking pin comprises locking pin threads positioned between a first end and the second end of the locking pin, the locking pin received by the threaded opening, wherein the knob is designed to rotate within the opening and selectively engage with the locking pin, and the locking pin threads selectively engage with the threaded opening, and wherein the bender fork translates within the recess between at least a first bender fork position and a second bender fork position.

According to a second aspect, the system of the first aspect or any other aspect, wherein the push rod further comprises an angled surface that selectively engages the prong.

According to a third aspect, the system of the second aspect or any other aspect, wherein the prong further comprises a separating boss that selectively engages the angled surface.

According to a fourth aspect, the system of the third aspect or any other aspect, wherein the prong further comprises an angled portion that selectively engages with the inner surface.

According to a fifth aspect, the system of the first aspect or any other aspect, further comprising a retention block, the retention block receiving a first portion of the surgical implant, the surgical implant forming a first implant orientation.

According to a sixth aspect, the system of the first aspect or any other aspect, further comprising a spacer having a first end, a second end, and at least one distribution wing, the first end of the spacer selectively engaging with the second end of the push rod.

According to a seventh aspect, the system of the sixth aspect or any other aspect, wherein one or both of the second end and the distribution wing of the spacer selectively engages with the surgical implant.

According to an eighth aspect, the system of the first aspect or any other aspect, wherein the hook selectively engages with a second portion of the surgical implant.

The present disclosure also relates to an inserter for inserting a surgical implant, the inserter comprising, according to a ninth aspect: a housing having a first end and a second end, the housing defining a hollow interior portion, the interior portion defined by an inner surface extending internally within the housing between the first end and the second end of the housing, a bender fork having a first end and a second end, the bender fork disposed within the housing, the bender fork comprising: a first threaded portion positioned at the first end of the bender fork, at least one prong and at least one hook disposed at the second end of the bender fork, wherein the at least one hook at least partially extends below the second end of the housing, a separating boss extending from each prong, and an angled portion selectively engaged with the inner surface, a push rod disposed within the housing and selectively engaged with the bender fork, the push rod comprising an angled surface selectively engaged with the angled portion of the bender fork, a locking pin disposed within the housing, the locking pin comprising a second threaded portion rotatably engaged with the first threaded portion, wherein rotation of the locking pin causes the bender fork to translate linearly with respect to the locking pin, and a knob secured to one end of the locking pin, wherein rotating the knob causes the locking pin to rotate.

According to a tenth aspect, the inserter of the ninth aspect or any other aspect, further comprising a spacer having a first end and a second end, the first end of the spacer selectively engaging with the second end of the push rod.

According to an eleventh aspect, the inserter of the tenth aspect or any other aspect, wherein the spacer further comprises at least one distribution wing.

The present disclosure also relates to a method for inserting or retrieving a surgical implant, the method comprising, according to an twelfth aspect: identifying a prepared surgical site within a patient; aligning an inserter assembly with the surgical site, the inserter assembly comprising: a housing having a first end and a second end and defining an interior compartment; a knob rotatably attached within the first end of the housing; a bender fork disposed within the housing, the bender fork comprising at least one prong, the at least one prong further comprising at least one hook extending from the second end of the housing, wherein the bender fork is designed to translate between a first bender fork position and a second bender fork position; and rotating the knob to translate the bender fork.

According to a thirteenth aspect, the method of the twelfth aspect or any other aspect, further comprising the step of loading the surgical implant within the inserter assembly, the surgical implant forming a first implant orientation.

According to a fourteenth aspect, the method of the thirteenth aspect or any other aspect, wherein the at least one hook selectively engages with the surgical implant.

According to a fourteenth aspect, the method of the thirteenth aspect or any other aspect, further comprising the step of rotating the knob to translate the bender fork from a first bender fork position to a second bender fork position.

According to a fifteenth aspect, the method of the fourteenth aspect or any other aspect, wherein the at least one prong transmits force to the at least one hook.

According to a sixteenth aspect, the method of the fifteenth aspect or any other aspect, wherein the surgical implant forms a second implant orientation.

According to a seventeenth aspect, the method of the sixteenth aspect or any other aspect, further comprising the step of inserting the surgical implant into the prepared surgical site.

According to an eighteenth aspect, the method of the seventeenth aspect or any other aspect, further comprising the step of rotating the knob to translate the bender fork from the second bender fork position to the first bender fork position.

According to a nineteenth aspect, the method of the eighteenth aspect or any other aspect, wherein the at least one hook disengages from the surgical implant.

According to a twentieth aspect, the method of the nineteenth aspect or any other aspect, further comprising the step of deploying the surgical implant into the prepared surgical site by disengaging the at least one hook from the surgical implant, the surgical implant forming a third implant orientation.

It will be understood by those skilled in the art that one or more aspects of this disclosure can meet certain objectives, while one or more other aspects can lead to certain other objectives. Various modifications to the illustrated embodiments will be readily apparent to those skilled in the art, and the generic principles herein can be applied to other embodiments and applications without departing from embodiments of the disclosure. Other objects, features, benefits, and advantages of the present disclosure will be apparent in this summary and descriptions of the disclosed embodiments, and will be readily apparent to those skilled in the art. Such objects, features, benefits, and advantages will be apparent from the above as taken in conjunction with the accompanying figures and all reasonable inferences to be drawn therefrom.

### BRIEF DESCRIPTION OF THE DRAWINGS

FIG. 1 is an isometric view of an exemplary insertion assembly with an implant in a first orientation, according to one embodiment;

FIG. 2 is a side view of an implant in a second orientation, according to one embodiment;

FIG. 3 is an isometric view of the implant of FIG. 2;

FIG. 4 is a side view of an implant in a third orientation, according to one embodiment;

FIG. 5 is an isometric view of the implant of FIG. 4;

FIG. 6 is an isometric view of a first component of the inserter assembly of FIG. 1, according to one embodiment;

FIG. 7 is an isometric view of a second component of an inserter assembly of FIG. 1, according to one embodiment;

FIG. 8 is an isometric view of a third component of the inserter assembly of FIG. 1, according to one embodiment;

FIG. 9 is an isometric cross-section view of FIG. 8;

FIG. 10 is an isometric view of a fourth component of the inserter assembly of FIG. 1, according to one embodiment;

FIG. 11 is an isometric view of a fifth component of the inserter assembly of FIG. 1, according to one embodiment;

FIG. 12 is an isometric view of a sixth component of the inserter assembly of FIG. 1, according to one embodiment;

FIG. 13 is an isometric view of a seventh component of the inserter assembly of FIG. 1, according to one embodiment;

FIG. 14 is a front view of an inserter assembly with an implant in a second orientation, according to one embodiment;

FIG. 15 is a side view of an inserter assembly and the implant of FIG. 14;

FIG. 16 is a side cross-section view of FIG. 15;

FIG. 17 is an isometric view of an inserter assembly with an implant in a second orientation, in relation to patient anatomy, according to one embodiment;

FIG. 18 is an isometric cross-section view of an inserter assembly with an implant in a second orientation, in relation to patient anatomy, according to one embodiment;

FIG. 19 is a side cross-section view of an inserter assembly with an implant in a third orientation, in relation to patient anatomy, according to one embodiment;

FIG. 20 is an isometric view of an inserter assembly with an implant in a third orientation, in relation to patient anatomy, according to one embodiment;

FIG. 21 is an isometric cross-section view of an inserter assembly with an implant in a third orientation, in relation to patient anatomy, according to one embodiment;

FIG. 22 is a flowchart of an exemplary method of inserting an implant using the inserter assembly, according to one embodiment; and

FIG. 23 is a flowchart of an exemplary method of removing an implant using the inserter assembly, according to one embodiment.

### DETAILED DESCRIPTION

Generally, surgical implants (“implants”) designed for fixation, such as orthopedic staples, can include two or more legs to address various anatomical/surgical requirements in different areas of patient anatomy when inserted into a target surgical site (e.g., a prepared portion of patient anatomy). Such implants can comprise shape memory or super-elastic materials that may be suitable for imparting compressive force on bone or other patient anatomy, which can promote the post-operative healing process by holding bones and tissues in a desirable position over time. In some cases, implants can include an implant bridge and two or more implant legs extending therefrom, wherein the legs of the implants are designed to form a relatively curved shape/contour in a first orientation (e.g., at rest or unstrained with the implant legs substantially converging) prior to insertion within a patient, then a deformed shape/contour in a second orientation (e.g., strained with the implant legs substantially parallel to or diverging from each other) in preparation for and during insertion, and then a reformed shape/contour in a third orientation (e.g., reduced strain with the implant legs substantially converging again) after insertion into the surgical site. Similarly, the bridges of the implants can form rest/unstrained, strained/deformed, and reformed shapes in the first, second, and third orientations, respectively.

In certain cases, the implants may be challenging to manually deform (or remain deformed once manually deformed) between the first, second, and/or third orientations, which may affect the variance in compressive force during insertion into or removal from patient anatomy as well as the amount of force applied to the implant bridge and/or legs. In some cases, the implants may be challenging to move from the first orientation to the second orientation, which may affect insertion into the surgical site. In other cases, the implants may be challenging to move from the third orientation to the second orientation, which may affect removal from the surgical site. For instance, the implants may be thermally sensitive, and placing the implant legs in the second orientation from the third orientation may apply undesirable forces to the implants after they have been maintained at body temperature for extended time periods. Additional challenges may be faced by removing implants having more than two implant legs (e.g., three or four implant legs) because such implants are designed to apply increased compressive forces within the surgical site, and, accordingly, may be more rigid and resistant to manual deformation.

Accordingly, briefly described, aspects of the present disclosure generally relate to an inserter assembly system designed to transport (e.g., store, deliver, insert, retrieve/remove) an implant to/from a surgical site involving bones or bone segments/bone resection areas. In particular, an inserter assembly is described herein for improving the opening and releasing of an implant having two or more implant legs, or another number of legs, without departing from the principles of this disclosure. In some embodiments, the inserter assembly is designed to maintain an implant in a first orientation during transportation, preparation, storage and/or delivery, and move the implant to a second orientation for insertion into the surgical site. To do so, the inserter assembly may supply a restraining or deforming force to portions of the implant via one or more components of the inserter assembly. In this way, the inserter assembly can generate a desirable amount of force to affect the orientation of the implant without damaging the implant. Then, once inserted and released into the surgical site, the implant may form a third orientation, thereby providing compression and fixation within the surgical site.

Additionally, it may be desirable to remove the implants once they have been inserted. For instance, the implants may become damaged upon initial insertion or over time, may not be properly inserted initially, or may not be bioresorbable or otherwise intended to remain within the patient long-term. Thus, in some cases, the inserter assembly can also be designed to remove the implant once installed within the surgical site. This may include circumstances wherein the implant is maintained at human body temperatures. To do so, one or more components of the inserter assembly can be designed to move the implant from the third orientation to the second orientation with minimal disruption to underlying patient anatomy around the surgical site and minimal damage to the implant.

In at least one embodiment, the inserter assembly comprises several components that facilitate improved storage, delivery, insertion, and/or removal of an implant. For instance, in some embodiments, the inserter assembly can include a retention block designed to maintain the implant in the first orientation by providing a recessed surface designed to receive a portion of the implant during storage. In some embodiments, the inserter assembly can include a housing designed to provide a surface for a user to grasp while transporting the implant using the inserter assembly. In some embodiments, the inserter assembly can include one or more bender forks (having at least two prongs) designed to selectively engage with or grasp the implant, thereby allowing the user to alter the orientation of the implant for insertion or retrieval from the surgical site. The bender forks may include hooked portions designed to selectively engage with an edge of the implant, thereby mechanically moving it to the second orientation from the first orientation.

In at least one embodiment, the inserter assembly includes further components such as an external knob designed to activate and deactivate one or more internal components of the assembly. In particular, the user can rotate the knob to convert rotational forces (torque) to linear translation of certain internal components that can cause the bender forks to engage and disengage with the implant. For instance, in some cases, rotating the knob may cause the bender forks to engage/grasp the implant, thereby moving the implant to the second orientation. In certain cases, rotating the knob may cause the bender forks to disengage/release the implant once the implant is inserted into the surgical site, thus allowing the implant to assume the third orientation. In other cases, rotating the knob may cause the bender forks to engage/grasp the implant once it has been inserted and move the implant legs into the second orientation for removal.

According to certain embodiments, as the user turns the knob to engage or disengage the implant, the bender forks may translate between at least a first bender fork position (e.g., extended from the housing) and a second bender fork position (e.g., retracted towards the housing) while the housing remains stationary relative to the bender forks. In some cases, the bender forks may translate to any suitable position between the first and second bender fork positions without departing from the principles of this disclosure.

In some embodiments, the inserter assembly may include a locking pin designed to rotate within the housing (with the knob) but resist translation. Certain features of the locking pin may selectively engage with certain other features of the bender forks, such that rotation of the locking pin can cause translation of the bender forks. Additionally, in some embodiments, the inserter assembly may include a push rod designed to remain stationary relative to the housing while the assembly is in operation, thereby providing counter-force in a linear direction for the bender forks. In some embodiments, the inserter assembly can include a spacer designed to protect (e.g., prevent marring) the implant as other components of the inserter assembly selectively engage with the implant. Further, the spacer may remain stationary relative to the housing to provide a surface against which the implant can selectively engage when the bender forks translate between the first and second bender fork positions (and thus the implant moves from the first orientation to the second orientation).

According to certain embodiments, various materials and material properties can be used to manufacture the various components of the inserter assembly. For instance, it may be desirable in certain situations for the inserter assembly or portions thereof to comprise materials that are suited to various imaging techniques, such that the components are at least partially radiolucent or radiopaque, according to user preferences. Exemplary materials include metals, plastics (e.g., biocompatible plastic), wood, composites, or any other suitable materials (e.g., other biocompatible materials), or combinations thereof. In some embodiments, the inserter assembly can comprise one or more materials to achieve patient-specific goals without departing from the principles of this disclosure. Furthermore, manufacturing techniques for the components of the inserter assembly may involve machining, injection molding, thermoforming or any other suitable manufacturing techniques or combinations thereof without departing from the principles of the disclosure.

According to various embodiments, the implant may comprise a shape memory or super-elastic material (e.g., nitinol), such that it can desirably store and release energy when deflected between the first, second, and third orientations. Thus, the implant can be designed to provide fixation after a surgical procedure for a patient. For example, the implant legs can be placed into the surgical site (e.g., a fractured or resected bone), wherein the implant bridge connects the implant legs at opposing sides of the implant bridge to provide mechanical strength and to retain any bone grafts used in the procedure. As will be understood, the implant can be manufactured in multiple ways, such as wire electro discharge machining, conventional machining, laser cutting, water jet cutting, or any other suitable manufacturing techniques or combinations thereof without departing from the principles of this disclosure.

The above features (and others) will be discussed herein in the context of an apparatus designed to insert or remove a shape memory fixation implant from a surgical site involving a bone fracture or resection area. However, it will be understood that the concepts discussed here are applicable to any suitable fixation and insertion/removal assembly system thereof used to support any human (or animal) anatomy without departing from the principles of this disclosure.

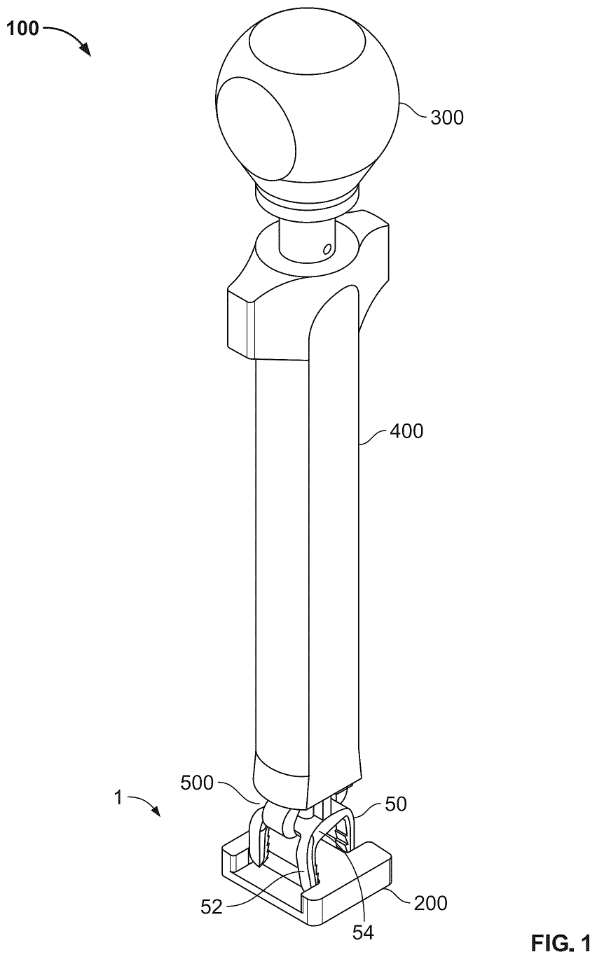

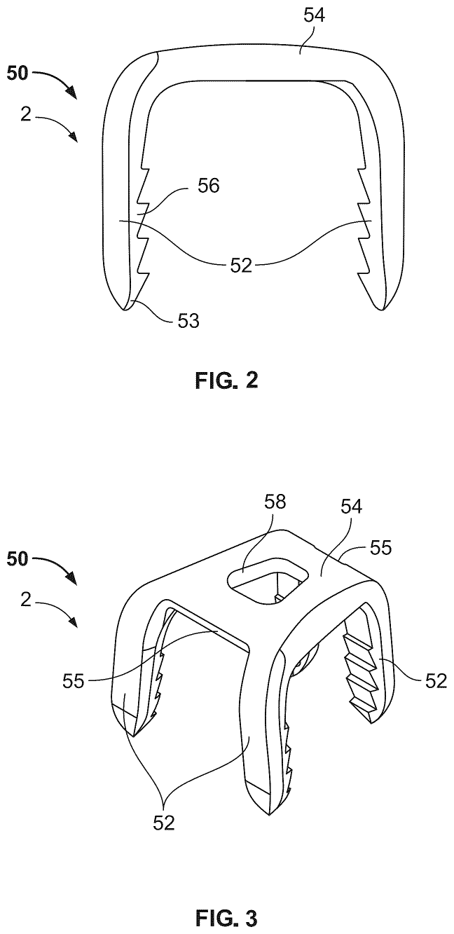

Turning now to FIG. 1, an isometric view of an exemplary inserter assembly 100 and an exemplary implant 50 are shown, according to one embodiment. Generally, the implant 50 can include at least an implant bridge 54 and two or more implant legs 52 (see FIGS. 2 and 3). Here, the implant 50 includes four implant legs 52, but other embodiments of the implant 50 can include at least two, at least three, at least five, or at least six or more implant legs 52. In some cases, a user can load the inserter assembly 100 with the implant 50, while in other cases, the inserter assembly 100 may be packaged and/or pre-loaded with the implant 50. In particular embodiments, the inserter assembly 100 may be sterilized together with the implant 50 (e.g., after the inserter assembly 100 has been loaded with the implant 50) while in other embodiments, the inserter assembly 100 and the implant 50 may be received sterilized.

Further, in some embodiments, the inserter assembly 100 includes one or more components designed to selectively engage with the implant 50. As shown, the one or more components of the inserter assembly 100 can engage with the implant 50 such that the implant 50 forms a first orientation 1. In some embodiments, the first orientation 1 involves the implant 50 forming a stored or rest state, wherein the implant legs 52 are naturally converging towards each other. In some cases, the implant 50 can form the first orientation 1 when it is loaded into the inserter assembly 100 while in other cases, the implant 50 can form the first orientation 1 without being loaded into the inserter assembly 100. As will be discussed herein, the one or more components of the inserter assembly 100 can maintain the implant 50 in its first orientation 1 as well as change the orientation of the implant 50 for transportation (involving delivery, insertion, and/or removal) of the implant 50 to and from a surgical site.

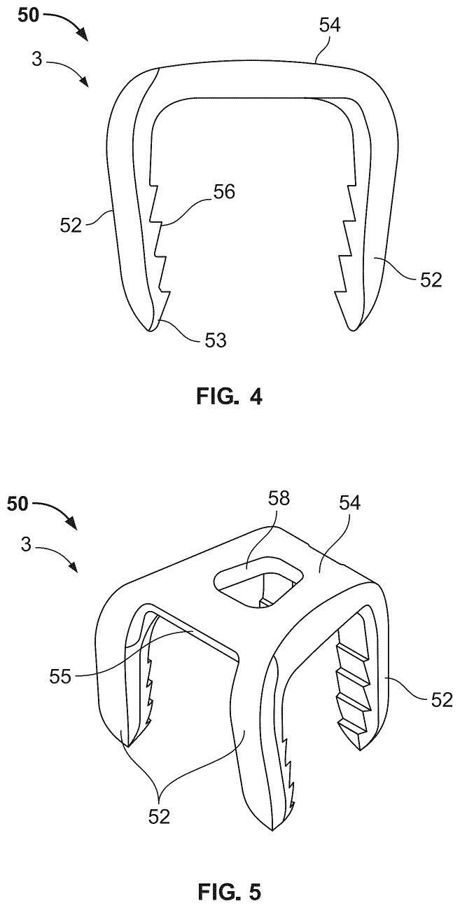

In some embodiments, the inserter assembly 100 can include components such as, but not limited to, a retention block 200, a knob 300, a housing 400, and one or more bender forks 500. In some cases, the knob 300, the housing 400, and the bender forks 500 can comprise a body of the inserter assembly 100 (with or without other components). According to particular embodiments, the user can grasp the housing 400 and engage (e.g., rotate) the knob 300 to arrange the implant 50 in different orientations. For instance, prior to turning the knob 300, the implant legs 52 can engage with and rest against the retention block 200 when the implant 50 forms the first orientation 1. In at least this way, the retention block 200 can hold/maintain the implant legs 52 in the first orientation 1 and protect the implant 50 from damage during transportation. Then, further engaging the knob 300 can cause the bender forks 500 to apply force to the implant bridge 54. In at least this way, the implant bridge 54 can transition between a convex or arch-like shape to a relatively straight shape or concave shape to move the implant legs 52 apart from the first orientation 1 to a second orientation 2 (see FIGS. 2 and 3), or from a third orientation 3 (see FIGS. 4 and 5) to the second orientation 2, or any other suitable orientation. Each component will now be explained in greater detail.

Turning now to FIGS. 2-5, various views and configurations of the implant 50 are shown. In many cases, the implant 50 may comprise a shape memory super-elastic implant having a naturally converging shape which can be modified over the course of a surgical procedure. Generally, the implant 50 can include an implant bridge 54 having one or more implant legs 52 each extending at an angle from opposing ends of the implant bridge 54. In certain embodiments, the one or more implant legs 52 can be sized to achieve different surgical outcomes. For instance, in certain cases, each of the one or more implant legs 52 may be sized relatively similarly, while in other cases, the one or more implant legs 52 may be sized relatively differently according to user preferences. In some embodiments, the one or more implant legs 52 may be provided with one or more barbs 56 (e.g., teeth-like structures) that are designed to improve grip and resist loosening/extraction after the implant 50 is installed within the surgical site. Further, the implant legs 52 may each comprise a tip 53 suited for insertion into a surgical site. In some cases, at least one of the tips 53 may include a tapered shape.

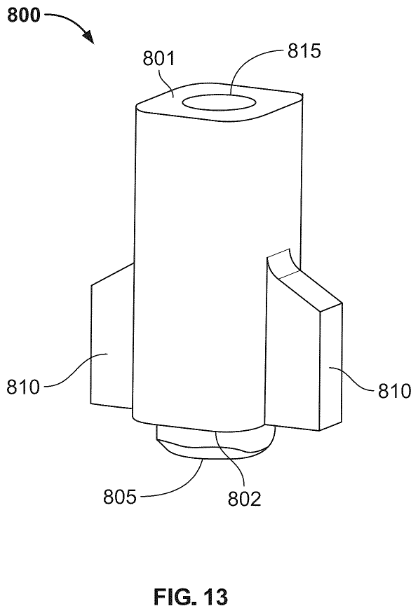

In certain embodiments, the implant bridge 54 may include a window 58 provided in the form of a through-hole opening. In some cases, the window 58 may allow the user to visualize underlying patient anatomy. In other cases, the window 58 may reduce the overall mass and/or material composition of the implant 50, thus allowing for a relatively lightweight implant 50. In other embodiments, the window 58 may be omitted from the implant bridge 54. In certain cases, the window 58 may be sized to receive one or more components of the inserter assembly 100 (e.g., spacer 800 of FIG. 13) when the inserter assembly 100 is in use.

Additionally, in some embodiments, the implant bridge 54 includes one or more edges 55 (see FIGS. 2 and 4) that the components of the inserter assembly 100 can grasp and/or apply force to in order to modify the orientation of the implant 50. This, in turn, can modify the orientation of the implant 50 between the first orientation 1 (e.g., converging, stored, rest), the second orientation 2 (e.g., strained, deformed), the third orientation 3 (e.g., at least partially converging, reformed), and any other orientation therebetween.

In particular, FIGS. 2 and 3 depict the implant 50 forming the second orientation 2, according to one embodiment. In certain embodiments, the second orientation 2 involves the implant 50 forming a deformed or strained state, wherein each of the implant legs 52 are relatively and substantially parallel with respect to each other. In some embodiments, the implant legs 52 may at least partially diverge from each other in the second orientation 2 without departing from the principles of this disclosure. In some cases involving the second orientation 2, the implant bridge 54 may form a substantially straight shape while in other cases, the implant bridge may form an at least partially concave shape. In some instances, the implant legs 52 can be moved from the first orientation 1 to the second orientation 2 by applying an arching or pulling force to the edges 55 of the implant bridge 54. Thus, while the implant 50 is in the second orientation 2, the altered orientations of the implant legs 52 and/or the implant bridge 54 may allow for the implant 50 to be inserted more easily and evenly into a surgical site.

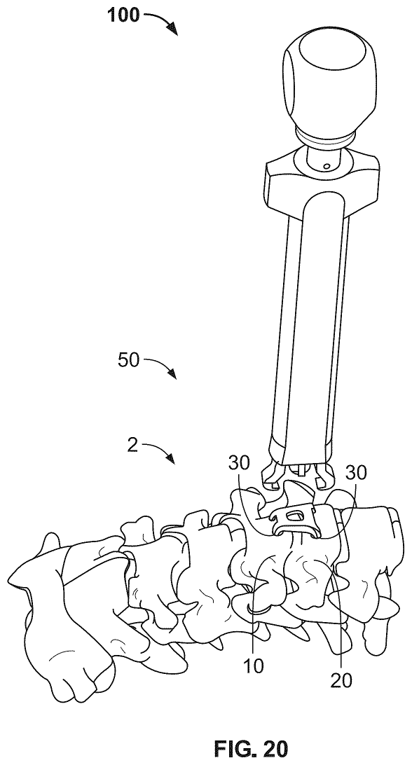

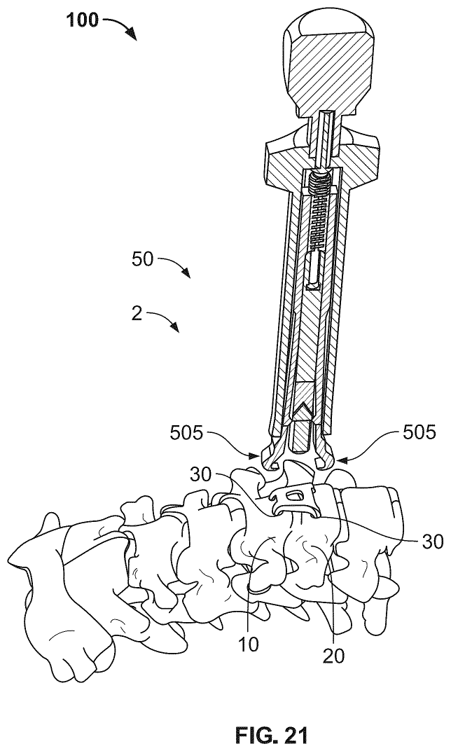

With particular reference to FIGS. 4 and 5, a side view and an isometric view are shown of the implant 50 in the third orientation 3, according to one embodiment. In some embodiments, the third orientation 3 involves the implant 50 forming a reformed configuration once the implant 50 is inserted into a surgical site (see FIGS. 20 and 21), wherein the implant legs 52 at least partially converge with respect to each other. In the third orientation 3, each of the implant legs 52 may be angled inwards towards each other. In some cases, only certain implant legs 52 may angle inward in order to achieve different surgical outcomes (e.g., certain implant legs 52 exhibit a first angle and certain other implant legs 52 exhibit a second angle). In some embodiments, by applying a pulling force on the one or more edges 55 of the implant bridge 54, the implant legs 52 can be moved from the third orientation 3 to the second orientation 2. In some cases, it may be desirable to stabilize a center portion of the implant bridge 54 (such that the implant bridge 54 is stationary) while moving the implant 50 from the third orientation 3 to the second orientation 2. For example, the implant legs 52 can be inserted into the surgical site (e.g., a fractured or resected bone), wherein the implant bridge 54 connects the implant legs 52 at opposing sides of the implant bridge 54 to provide mechanical strength and to retain any bone fragments or grafts used in the procedure.

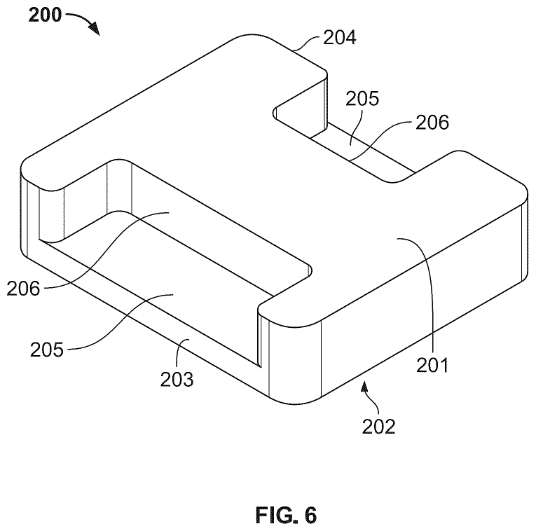

Turning now to FIG. 6, an isometric view of the retention block 200 is shown, according to one embodiment of the present disclosure. The retention block 200 can be provided in the form of a body including a first surface 201, a second surface 202, a first side 203, and a second side 204. In certain embodiments, the retention block 200 can include one or more recesses 205 positioned on the first surface 201 at the first side 203 and the second side 204. The recesses 205 can be designed to receive at least a portion of the implant legs 52. For instance, in some embodiments, the recesses 205 can receive the tips 53 of the implant legs 52 while the implant 50 forms the first orientation 1. As shown, the retention block 200 can include two recesses 205, although in other embodiments, the retention block 200 can include any suitable number of recesses 205 to correspond with the implant legs 52. For instance, the retention block 200 can be provided with three, or four, or five, or six or more recesses 205 without departing from the principles of this disclosure.

In some embodiments, the retention block 200 can be designed to hold or maintain the implant legs 52 in a desirable orientation before and during a surgical procedure. Accordingly, the user can selectively engage the tips 53 of the implant legs 52 with interior portions 206 of the recesses 205 to position the implant 50 and the implant legs 52 in the first orientation 1. In this way, the retention block 200 can be designed to relieve the implant 50 and/or the inserter assembly 100 from undesirable forces during storage and transport of the implant 50. In other embodiments, the retention block 200 can be designed to supply a partially restraining force to the implant 50, which can support loads and prevent binding of the implant 50 to other components of the inserter assembly 100.

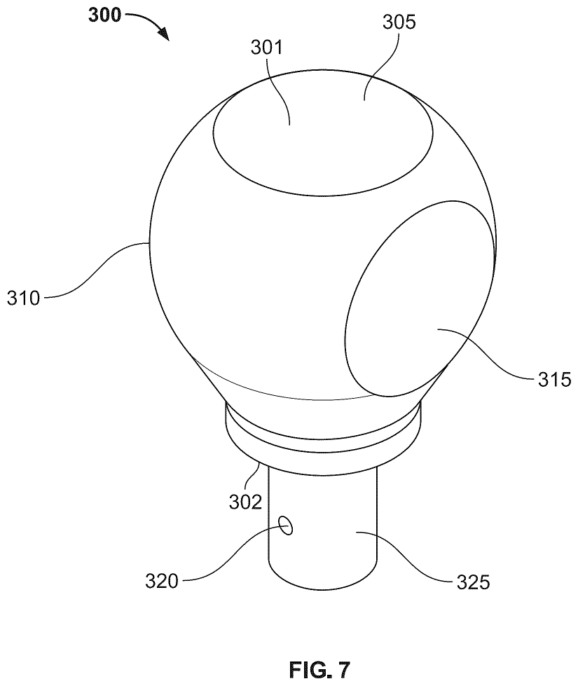

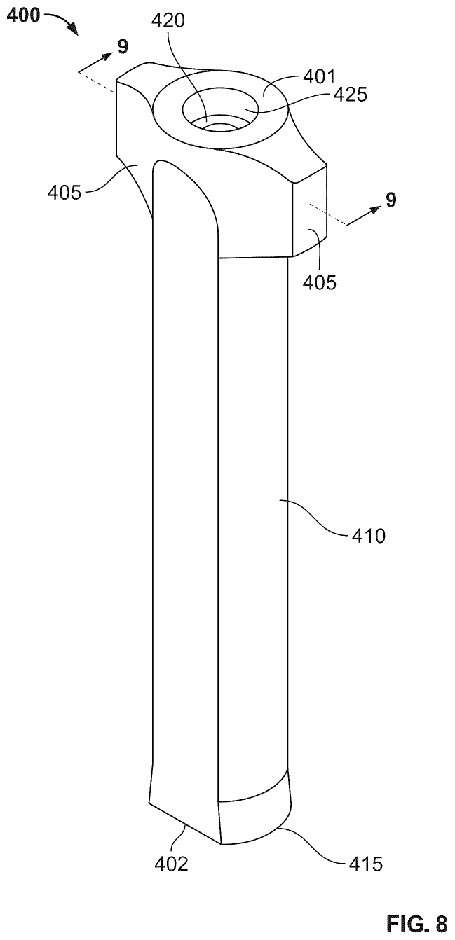

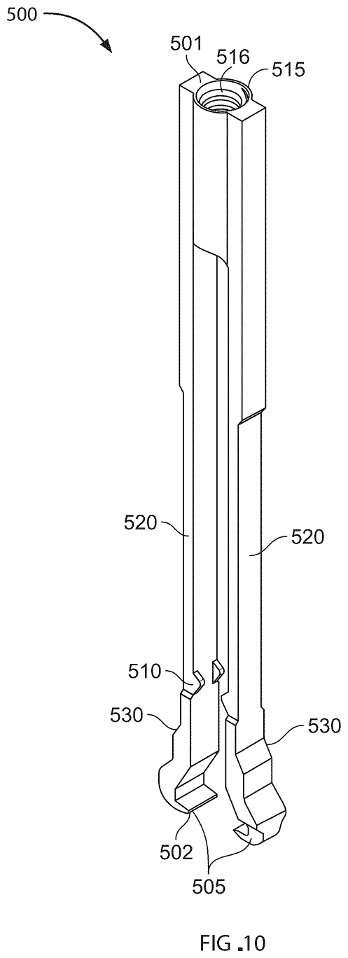

Turning now to FIG. 7, an isometric view of the knob 300 is shown, according to one embodiment. The knob 300 can be provided in the form of a body 310 having a substantially spherical or curved shape and having a substantially smooth surface designed for user comfort, but may include alternative shapes or textures without departing from the principles of this disclosure. In particular, the knob 300 can be attached to or within the housing 400 such that the user can manually engage with the knob 300 to operate the inserter assembly 100. For instance, rotating or turning the knob 300 in a first direction (e.g., clockwise) or a second direction (e.g., counter-clockwise) can transform rotational force provided by the user to bring about linear translation/motion of the bender forks 500 (see FIGS. 1 and 10). According to particular embodiments, the knob 300 in combination with one or more other features of the inserter assembly 100 (e.g., fins 405 shown in FIG. 8) may allow the user to manually rotate the knob 300 in the first or second directions while grasping the housing 400.

In some embodiments, the knob 300 includes a body 310 having a first end 301 and a second end 302, wherein the body 310 can be provided with one or more features adapted for manual interaction by the user. For instance, in some embodiments, the body 310 can include an impact surface 305 positioned at the first end 301 to provide a surface for the user to impact the overall inserter assembly 100 when installing the implant 50. In some cases, the user may use a hammer or any other suitable tool to impact the impact surface 305. In some embodiments, the body 310 can include one or more gripping portions 315 positioned between the first end 301 and the second end 302 to provide substantially flat areas along the body 310 suited for improved manual interaction by the user. In some cases, this may allow the user to generate increased torque when turning the knob 300. In other cases, this may allow the user to more precisely turn the knob 300 while using the inserter assembly 100.

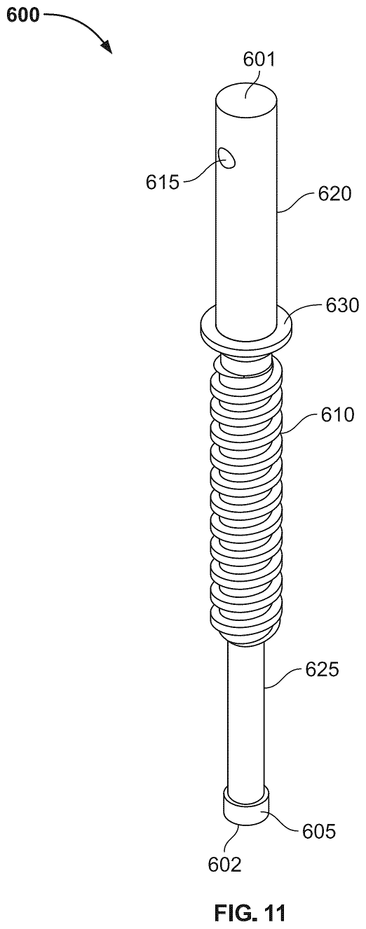

Further, in some embodiments, a tubular shaft 325 can extend from the second end 302 of the body 310, wherein the tubular shaft 325 can be received by a portion of the housing 400 (see FIG. 1). In this way, the tubular shaft 325 provides an attachment point between the knob 300 and other components of the inserter assembly 100. In some embodiments, the tubular shaft 325 can include a first fixation hole 320 sized to receive a fixation pin (not shown), wherein the fixation pin can removably attach the knob 300 with at least one end of a locking pin 600 (see FIG. 11) when inserted through the first fixation hole 320. For instance, when assembling the inserter assembly 100, a second fixation hole 615 (see FIG. 11) positioned on the locking pin 600 may be aligned with the first fixation hole 320 positioned on the tubular shaft 325, and a fixation pin can be inserted through each of the fixation holes 320, 615 to attach the knob 300 to the locking pin 600. In this way, when the user rotates the knob 300, the locking pin 600 may also rotate. In some embodiments, the fixation pin can be provided in the form of a nail, screw, dowel, or any other suitable method of fixation/attachment component without departing from the principles of this disclosure. Alternatively, the knob 300 may be attached to any other suitable component of the inserter assembly 100 via any suitable attachment method such as, but not limited to, temporary fixation (e.g., via the fixation pin), adhesion, welding, or any other method or suitable combination thereof.

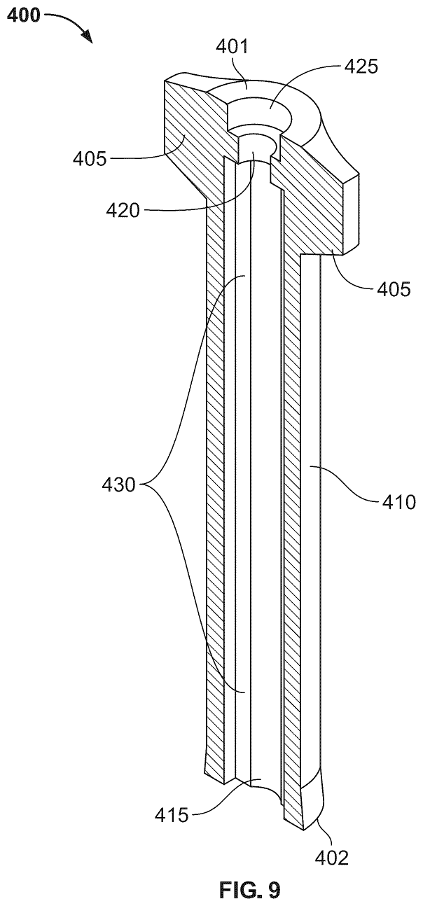

In embodiments described herein, various features of the one or more components of the inserter assembly 100 may reduce or prevent the knob 300 from linear translation relative to the housing 400 (see FIG. 1). For instance, an upper opening 425 (see FIG. 8) of the housing 400 may be sized to receive the tubular shaft 325. In this way, the upper opening 425 may selectively engage with the second end 302 and thus prevent translation of the knob 300 when the inserter assembly 100 is assembled. In further embodiments, an inner ring 420 disposed within the housing 400 (see FIG. 9) may be sized to receive a stop ring 630 positioned on the locking pin 600 (see FIG. 11), thereby preventing translation of the locking pin 600. Because the knob 300 may be removably attached to the locking pin 600 (via first and second fixation holes 320, 615 and a fixation pin), the locking pin 600 can reduce or prevent translation of the knob 300. Thus, in certain embodiments, the knob 300 can be rotated and cause rotation of the locking pin 600, but the knob 300 may resist translation relative to the housing 400.

Turning now to FIG. 8, an isometric view of a housing 400 is shown, according to one embodiment. In particular, the housing 400 can be provided in the form of an exterior shell or housing designed to protect and secure the internal components of the inserter assembly 100. Further, the housing 400 can remain relatively stationary and provide the user with additional gripping surfaces when the user operates the inserter assembly 100, thereby providing counter-torque when the user turns the knob 300. As shown, the housing 400 comprises essentially a hollow shaft 410 having a first end 401 and a second end 402. In some embodiments, the hollow shaft 410 can include one or more fins 405 positioned proximal the first end 401 (and thus, the knob 300) to provide gripping surfaces allowing the user to generate increased torque. In particular, the user can apply counter-torque to the housing 400 by gripping the fins 405 or any other suitable portion of the housing 400. In this way, translational forces on the housing 400 may be reduced. Thus, when the user inserts the implant 50 into the surgical site using the inserter assembly 100, the housing 400 can remain relatively stationary in relation to the implant 50. As will be understood, in certain embodiments, the housing 400 may define at least one interior compartment for housing one or more components of the inserter assembly 100.

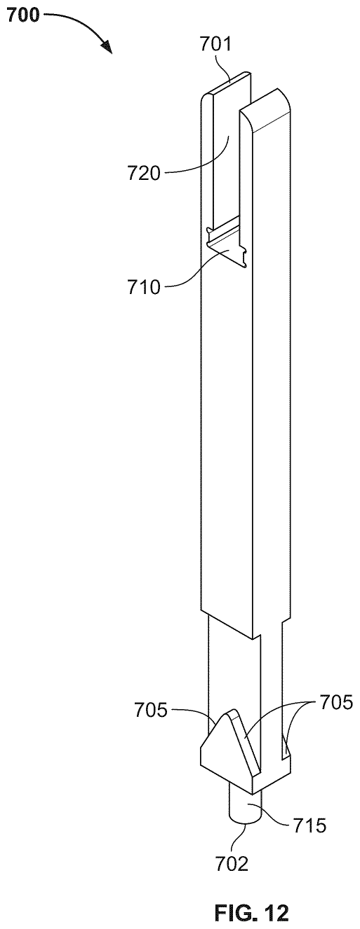

With reference to FIG. 9 (an isometric section view of the housing 400), the interior portion of the housing 400 can comprise an inner surface 430 having an inner recess 415. In one or more embodiments, the inner surface 430 can be provided in the form of a hollow, interior compartment within the hollow shaft 410. In some cases, the inner surface 430 can form a relatively round shape while in other cases, the inner surface 430 can form a relatively square or rectangular shape. Additionally, in some embodiments, the inner surface 430 can be sized to receive the bender forks 500 (see FIG. 10), the locking pin 600 (see FIG. 11), and/or a push rod 700 (see FIG. 12). In some cases, the inner recess 415 may form a portion of the inner surface 430 such that the inner recess 415 can be provided in the form of an interior depression disposed on a surface of the inner surface 430. In certain cases, the inner recess 415 includes a longitudinal axis substantially parallel to a longitudinal axis of the inner surface 430. In other cases, at least a portion of the inner recess 415 includes a tapered shape which may allow portions of the bender forks 500 to move inwards or outwards (e.g., due to portions of the bender forks 500 selectively engaging with the inner recess 415). In some embodiments, the inner recess 415 can selectively engage with or otherwise receive a portion of the bender forks 500 as the bender forks 500 translate along the inner surface 430 to selectively engage with the locking pin 600.

In some embodiments, the interior portion of the housing 400 can include an upper opening 425 positioned towards the first end 401. The upper opening 425 can be sized to receive or otherwise contain a portion of the tubular shaft 325 of the knob 300 (see FIG. 7). In some embodiments, the housing 400 can include an inner ring 420 provided in the form of an opening within the housing 400 and having a smaller diameter than the inner surface 430. The inner ring 420 can be designed to separate the inner recess 415 from the upper opening 425, thereby separating the bender forks 500 from the knob 300. In particular, the inner ring 420 can form a relatively flat surface for the locking pin 600 and the knob 300 to press against. In some embodiments, a fixation pin can removably attach the housing 400, the locking pin 600, and/or the knob 300 together to form a rigid assembly.

Turning now to FIG. 10, an isometric view of the bender forks 500 is shown, according to one embodiment. In some cases, the bender forks 500 may be disposed within and selectively engage with the inner recess 415 such that the bender forks 500 may include a longitudinal axis substantially parallel to the longitudinal axis of the inner recess 415. Generally, the bender forks 500 can selectively engage with portions of the implant 50, thus moving the implant 50 into the first orientation 1, the second orientation 2, the third orientation 3, or any other suitable orientation therebetween. In particular, while the user operates the inserter assembly 100, the bender forks 500 can translate between at least a first bender fork position and a second bender fork position within the inner surface 430. In some embodiments, the first bender fork position may involve the bender forks 500 extending from the housing 400, and the second bender fork position may involve the bender forks 500 retracting within or towards the housing 400. In other embodiments, the first and second bender fork positions can involve any other suitable positions therebetween. In various embodiments, while the bender forks 500 can either translate to the first bender fork position or to the second bender fork position relative to the knob 300, the housing 400, and/or the locking pin 600, the bender forks 500 may resist rotational motion. In some aspects, translation of the bender forks 500 between the first bender fork position and the second bender fork position may be linear with respect to other components of the inserter assembly 100 (e.g., the housing 400, the locking pin 600, the push rod 700 of FIG. 12).

In some embodiments, the bender forks 500 can include a first end 501 and a second end 502, each including further additional features. For instance, in some cases, the bender forks 500 may comprise one or more prongs 520 extending from the first end 501 towards the second end 502. The prongs 520 can be provided in the form of relatively flat surfaces. The prongs 520 can include outer surfaces that selectively engage with (e.g., contact) inner portions of the inner surface 430 (see FIG. 10) to, at least in part, prevent rotation of the bender forks 500 relative to the housing 400 while the inserter assembly 100 is in use. Further, the prongs 520 can include inner surfaces that selectively engage with certain portions of the push rod 700 (see FIG. 12). In certain cases, the prongs 520 may be biased outwards to push against the inner surface 430, thus transmitting force (generated by the user) to one or more hooks 505 that form end portions of each of the prongs 520. In certain embodiments, via the prongs 520 moving away from each other and/or moving towards each other, the hooks 505 may apply force to the implant 50 to shift the implant 50 between the first, second, and/or third orientations 1, 2, 3.

In some embodiments, the hooks 505 can be positioned at the second end 502 of the bender forks 500 and can be configured to extend at least partially outwards from the second end 402 of the housing 400. Thus, in some cases, the hooks 505 are positioned to selectively engage with the one or more edges 55 of the implant bridge 54 (see FIGS. 2 and 4). In some embodiments, the hooks 505 can apply a force to the edges 55 such that the implant legs 52 move between the first, second, and third orientations 1, 2, 3. Additionally, in some embodiments, the hooks 505 may be sized according to user preferences, implant 50 shape and size, and other patient-specific considerations.

In some embodiments, the bender forks 500 can include separating bosses 510 positioned near the first end 501. The separating bosses 510 can provide controlled separation of and prevent unintentional interaction or damage to components of the inserter assembly 100 while the inserter assembly 100 is in use. In some cases, the separating bosses 510 can provide controlled separation between the bender forks 500 and the push rod 700 (see FIG. 12). In particular, as the bender forks 500 transition to the second bender fork position, the separating bosses 510 may selectively engage with angled surfaces 705 of the push rod 700, thereby pushing the prongs 520 apart. In this way, the hooks 505 can selectively engage with the bridge 54 of the implant 50. Thus, the bender forks 500 can apply a pulling force such that the implant 50 interfaces with the spacer 800 (see FIG. 13), thereby creating a bend force to bend the implant 50 into the second orientation 2. In some cases, in combination with other components of the inserter assembly 100 (e.g., spacer 800), the bend force may be applied as a three-point bend force (e.g., at the edges 55 and a center portion of the implant bridge 54).

In some embodiments, the bender forks 500 can include angled portions 530 positioned near the second end 502. The angled portions 530 can selectively engage with (e.g., press against) the inner recess 415 or other interior portions of the housing 400 when the bender forks 500 translate between the first bender fork position (e.g., extended) and/or the second bender fork position (e.g., retracted). In this way, the angled portions 530 can cause the prongs 520 to move away from each other when the bender forks 500 translate to the second bender fork position. Furthermore, the angled portions 530 can cause the prongs 520 to move towards each other when the bender forks 500 translate to the first bender fork position, such that the hooks 505 can disengage from the bridge 54 of the implant 50. Thus, the separating bosses 510 may interface (e.g., at least partially disengage) with the angled surfaces 705, and the implant 50 can reform in orientation (e.g., second orientation 2, third orientation 1).

In some embodiments, the first end 501 includes an opening 516 sized to receive the locking pin 600. Further, the opening 516 can include bender fork threads 515, provided in the form of a female threaded surface arranged within the opening 516. In some cases, the bender fork threads 515 may be designed to complement a male threaded surface of the locking pin 600 (see, e.g., locking pin threads 610 of FIG. 11). As the user turns the knob 300 to rotate the locking pin 600, the locking pin threads 610 selectively or rotatably engage with the bender fork threads 515 to cause translation of the bender forks 500 between the first bender fork position and the second bender fork position. Thus, in certain embodiments, rotation of the locking pin 600 can cause the bender forks 500 to translate linearly with respect to the locking pin 600. In some embodiments, the bender fork threads 515 (and thus the locking pin threads 610) may exhibit a relatively fine thread pattern in order to increase precision in translating the bender forks 500, and thus increase precision in applying force to the implant bridge 54 via the hooks 505. In other embodiments, any other characteristics of the bender forks 500 may be adapted to affect the precision in applying force to portions of the implant 50.

Turning now to FIG. 11, an isometric view of the locking pin 600 is shown, according to one embodiment. In some embodiments, the locking pin 600 may be provided as a unitary component, while in other embodiments, the locking pin 600 may comprise one or more components. For instance, the locking pin 600 and the push rod 700 (see FIG. 12) may form a unitary component. In other instances, the locking pin 600 and the knob 300 may form a unitary component. The locking pin 600 can be designed to bring about translation of the bender forks 500 as the user rotates the knob 300. The locking pin 600 includes a first end 601 and a second end 602, each including further additional features. For instance, a portion of the locking pin 600 can be received by the opening 516 of the bender forks 500 such that the locking pin 600 may include a longitudinal axis substantially parallel to the longitudinal axis of the bender forks 500. In some embodiments, the locking pin 600 can include locking pin threads 610 positioned between the first end 601 and the second end 602. The locking pin threads 610 can selectively/rotatably engage with the bender fork thread 515 (see FIG. 10).

In some embodiments, a tubular shaft 625 can extend from the second end 602, wherein the tubular shaft 625 can be received by a portion of the push rod 700 (see FIG. 12). In this way, the tubular shaft 625 provides an attachment point between the locking pin 600 and other components of the inserter assembly 100. In some embodiments, the tubular shaft 625 can include a push ring 605 positioned at the second end 602, wherein the push ring 605 may be provided in the form of a ring-shaped extrusion disposed around the second end 602. In some cases, the push ring 605 may be received within a first cavity 710 of the push rod 700 (see FIG. 12). Additionally, the tubular shaft 625 may be received by the second cavity 720 of the push rod 700 (see FIG. 12). In this way, as the user turns the knob 300 and rotates the locking pin 600, the locking pin threads 610 can engage with the bender fork threads 515 to translate the bender forks 500 from the first bender fork position to the second bender fork position. In one non-limiting example, rotation of the locking pin threads 610 can cause complementary pulling or pushing to the bender fork threads 515. In this way, the bender forks 500 may be drawn into the housing 400 at the second bender fork position or extend from the housing 400 at the first bender fork position.

In some embodiments, a cylindrical shaft 620 can extend from the first end 601, wherein the cylindrical shaft 620 can be received by a portion of the knob 300, such as the tubular shaft 325 (see FIG. 7). In certain embodiments, a diameter of the tubular shaft 325 may be larger than a diameter of the cylindrical shaft 620. In this way, the cylindrical shaft 620 provides an attachment point between the locking pin 600 and the knob 300. Additionally, the cylindrical shaft 620 can include a second fixation hole 615. In some cases, a first fixation hole 320 positioned on the tubular shaft 325 may be aligned with the second fixation hole 615, and a fixation pin can be inserted through each of the first and second fixation holes 320, 615 to attach the locking pin 600 to the knob 300. In this way, the cylindrical shaft 620 can be removably attached with the tubular shaft 325. Thus, when the user rotates the knob 300, the locking pin 600 may also rotate. In one non-limiting example, a fixation pin can be inserted through the second fixation hole 615 and the first fixation hole 320 to align/stabilize the knob 300 with the locking pin 600 within the inserter assembly 100. In another non-limiting example, rotating the knob 300 may transmit force to the locking pin 600 via the fixation pin, which can cause rotation of the locking pin threads 610 that transmits force to the bender fork threads 515.

Additionally, the cylindrical shaft 620 can include a stop ring 630 positioned at the first end 601 (next to the locking pin threads 610). The stop ring 630 may have a larger diameter than the inner ring 420. Further, the stop ring 630 may selectively engage with or press against a portion of the inner ring 420 of the sleeve (see FIG. 9) while the inserter assembly 100 is in use. In this way, the stop ring 630 may allow for rotation of the locking pin 600 but prevent it from translating within the housing 400.

Turning now to FIG. 12, an isometric view of a push rod 700 is shown, according to one embodiment. In some cases, the push rod 700 may be disposed within the housing 400 to provide internal stability as other components of the inserter assembly 100 move. For instance, the push rod 700 may remain stationary relative to the rotating locking pin 600 and the translating bender forks 500. Further, at least a portion of the push rod 700 can selectively engage with at least a portion of the bender forks 500. In some cases, an outer portion of the push rod 700 can selectively engage with inner surfaces of the prongs 520 (see FIG. 9). In certain cases, the push rod 700 may include a longitudinal axis substantially parallel to the longitudinal axis of the bender forks 500. In some instances, the push rod 700 can be provided as a unitary component, while in other cases, the push rod 700 and the locking pin 600 can be provided as a unitary component.

As shown, the push rod 700 can include a first end 701 and a second end 702, each including additional features. For instance, in some embodiments, the first end 701 can include the first cavity 710 and the second cavity 720. The first cavity 710 can be sized to receive the push ring 605 (see FIG. 11), and the second cavity 720 can be sized to receive the tubular shaft 625 (see FIG. 11). In certain cases, the push ring 605 may rotate within the first cavity 710 while the inserter assembly 100 is in use. In some embodiments, the second end 702 can include one or more angled surfaces 705. In some cases, the one or more angled surfaces 705 can selectively engage with the separating bosses 510 of the bender forks 500 (see FIG. 10) as the bender forks 500 translate between a first bender fork position and a second bender fork position via the user rotating the knob 300. Further, while the bender forks 500 translate, the separating bosses 510 can selectively engage with or push against the (relatively stationary) angled surfaces 705. In turn, the outwards movement of the separating bosses 510 may force the prongs 520 apart to press against the inner surface 430 (see FIG. 9).

In other words, in certain embodiments, turning the knob 300 may cause the prongs 520 to extend or retract relative to the housing 400. This may allow the hooks 505 to engage with or disengage from the implant 50, thus grasping or releasing the implant 50. The second end 702 may further include, in some embodiments, an attachment feature 715. The attachment feature 715 may extend from the second end 702 and may be received by an opening 815 disposed on the spacer 800 (see FIG. 13). Further, in some cases, the locking pin threads 610 rotatably engaging with the bender fork threads 515 may translate the push rod 700 from a first push rod position (e.g., upwards) to a second push rod position (e.g., downwards), while in many other cases, the push rod 700 may remain relatively stationary relative to the housing 400 to provide stability for the other components of the inserter assembly 100.

Turning now to FIG. 13, an isometric view of a spacer 800 is shown, according to one embodiment. As shown, the spacer 800 includes a first end 801 and a second end 802 such that the spacer 800 can selectively engage with the push rod 700 at the first end 801 and can selectively engage with the implant 50 at the second end 802. In some cases, the spacer 800 and the push rod 700 may be provided as a unitary component, while in other cases, the spacer 800 and the push rod 700 may be provided as separate components. In some embodiments, the spacer 800 may provide a relatively static surface for the implant 50 while the inserter assembly 100 is in use. In certain embodiments, a position feature 805 of the spacer 800 may selectively engage with a portion of the implant bridge 54, such as the window 58. Thus, the spacer 800 may be positioned to provide a downward counter-force to the implant bridge 54 as the implant 50 shifts between the first orientation 1 and the second orientation 2. In this way, the spacer 800 can protect the implant bridge 54 from damage or other undesirable contact from other components of the inserter assembly 100. Additionally, in some embodiments, the spacer 800 may be held relatively stationary by the push rod 700 while the inserter assembly 100 is in use via the attachment feature 715 and opening 815.

In particular embodiments, the first end 801 can include an opening 815 that may selectively engage the attachment feature 715. The opening 815 may be sized to receive the attachment feature 715 of the push rod 700 (see FIG. 11). In some cases, the opening 815 can provide a point of attachment for the attachment feature 715, thereby stabilizing the push rod 700 and the spacer 800 with respect to each other. In some cases, the attachment feature 715 can selectively engage with the opening 815. In other cases, the attachment feature 715 and the opening 815 can be attached via adhesion or any other suitable means of attachment without departing from the principles of this disclosure.

In some embodiments, a position feature 805 can extend from the second end 802, wherein the position feature 805 can be provided in the form of a protrusion. The position feature 805 may be sized to selectively engage with the window 58 of the implant 50. In some cases, the position feature 805 may be shaped to at least partially conformally engage with a portion of the implant bridge 54. In some cases, as the hooks 505 engage with the edge 55 of the implant bridge 54 to shift the implant legs 52 from the first orientation 1 to the second orientation 2, the position feature 805 may selectively engage with the implant bridge 54, thereby providing counter-force to the implant bridge 54. In these embodiments (and others), the spacer 800 can provide counter-force to the implant bridge 54 and which is supplied by translation of the bender forks 500 in relation to the housing 400, which may bring the implant 50 into a temporary contact with the spacer 800. Additionally, in some embodiments, the second end 802 can include distribution wings 810 designed to interface with certain other portions of the implant 50, thus improving the distribution of engagement forces while the inserter assembly 100 is in use. In certain embodiments, distribution wings 810 extend across the implant bridge 54 such that the wings distribute the downward counter-force to the implant bridge 54 as the implant 50 shifts between the first orientation 1 and the second orientation 2. In such embodiments, the distribution wings 810 create a fulcrum around which the implant bridge 54 can bend.

In some embodiment, spacer 800 is made of a non-marring material and may be replaced as needed by the user. In certain other embodiments, spacer 800 is integrally formed with push rod 700, without departing from the principles of this disclosure. In still further embodiments, the spacer 800 may be integrally formed with push rod 700 and further include certain portions that are made of a non-marring material.

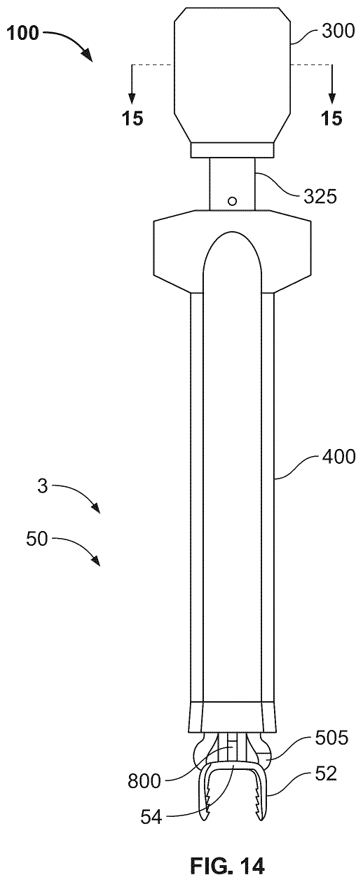

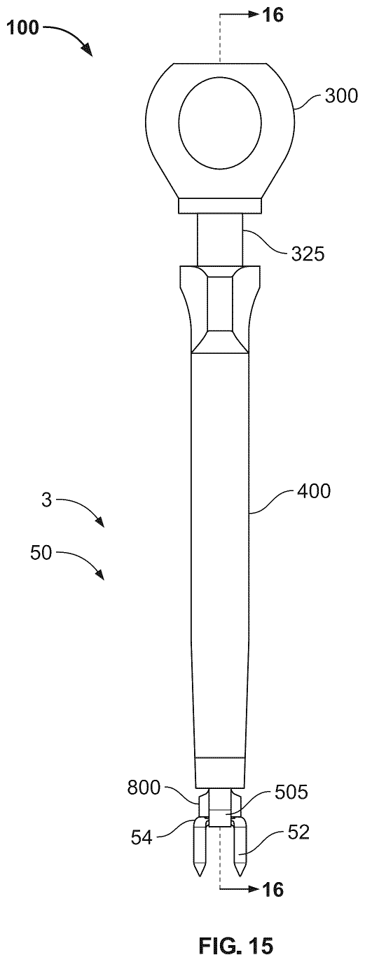

Turning now to FIGS. 14 to 20, additional views of the inserter assembly 100 are shown in operation, according to various embodiments. In particular, FIGS. 14 and 15 show side views of the inserter assembly 100, with the implant 50 in the second orientation 2, according to one embodiment. Here, the inserter has been assembled and positioned such that the bender forks 500 have translated from the first bender fork position (e.g., extended) to the second bender fork position (e.g., retracted). In this way, the hooks 505 may apply force to or pull at the edges 55 of the implant bridge 54 such that the implant 50 forms the second orientation 2. Meanwhile, the spacer 800 may remain relatively stationary to the implant 50 and provide a downward force against a center portion of the implant bridge 54 as the implant bridge 54 is pulled by the hooks 505, thereby causing the implant legs 52 to move apart to the second orientation 2 (e.g., wherein the implant legs 52 are relatively parallel or partially diverging with respect to each other). In this embodiment, once the inserter assembly 100 has moved the implant 50 from the first orientation 1 to the second orientation 2, the retention block 200 may be disconnected from the implant 50 without the user applying additional manual force (e.g., the user may easily remove the retention block 200 from the assembly 100).

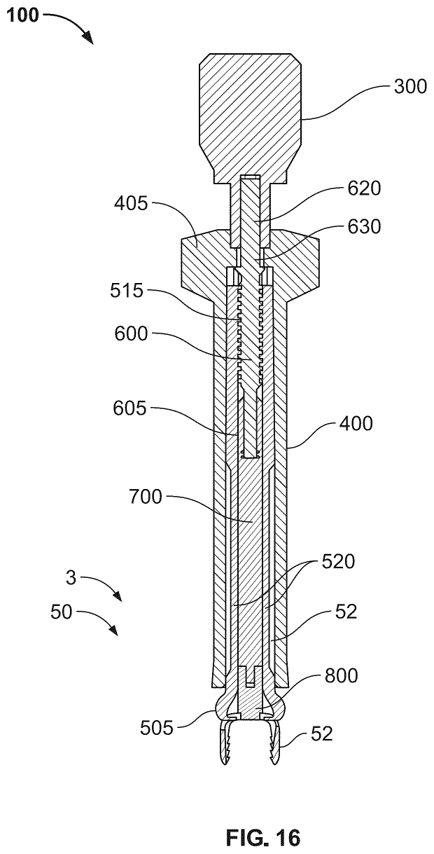

FIG. 16 shows a side section view of the inserter assembly 100, with the implant 50 in the second orientation 2, according to at least one embodiment. In some cases, further turning the knob 300 may rotate the locking pin 600. The rotating locking pin 600 may engage with the bender fork threads 515 via the locking pin threads 610 to cause translation of the bender forks 500 within the housing 400 while the push rod 700 and the spacer 800 remain relatively static. Further, in some embodiments, as the bender forks 500 translate within the housing 400, the angled portions 530 may interface with portions of the inner surface 430. In this way, the prongs 520 may be prevented from moving outward relative to each other, thus maintaining selective engagement between the hooks 505 and the implant bridge 54.

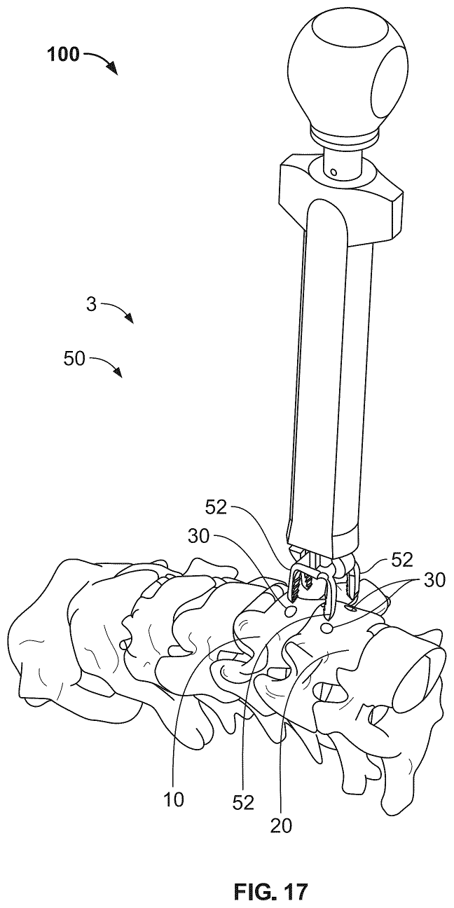

FIG. 17 shows an isometric view of the inserter assembly 100 approaching a surgical site, with the implant 50 in the second orientation 2, according to one embodiment. Here, the surgical site is depicted as at least two bone portions, shown in the form of a first bone portion 10 and a second bone portion 20. The first bone portion 10 and the second bone portion 20 can be any two bones or bone fragments/segments within the underlying patient anatomy. In one non-limiting example, the first bone portion 10 may represent one vertebra of the patient's spine, and the second bone portion 20 may represent a second adjacent vertebra of the spine. In some cases, each of the first bone portion 10 and the second bone portion 20 may include openings 30. The openings 30 may be drilled or impacted into the respective bone portions in order to improve insertion of the implant 50 into the surgical site. Additionally, the openings 30 may be positioned to map to the positions of the implant legs 52 in the second orientation 2.

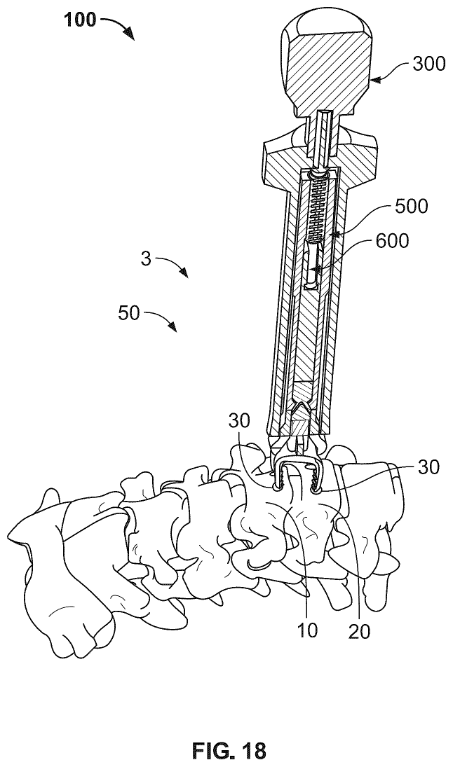

FIG. 18 shows a side section view of the inserter assembly 100, offset from a center, with the implant 50 in the second orientation 2 as it is being inserted into the surgical site, according to one embodiment. Here, the user may position the implant 50 in a desirable position within the surgical site, and begin the turn the knob 300 to cause the bender forks 500 to move toward the first bender fork position and begin the release the implant 50 into the surgical site. In at least one embodiment, the inserter assembly 100 may grasp the implant 50 perpendicularly to the first bone portion 10 and the second bone portion 20. In various embodiments, during insertion of the implant legs 52 into the prepared surgical site, certain components of the insertion assembly 100 may be relatively stationary (e.g., the implant 50, the knob 300, the bender forks 500, the locking pin 600, etc.). In certain cases, the inserter assembly 100 may be manipulated by the user to achieve any suitable combination of angles and orientations to position the implant 50 before, during, and after insertion.

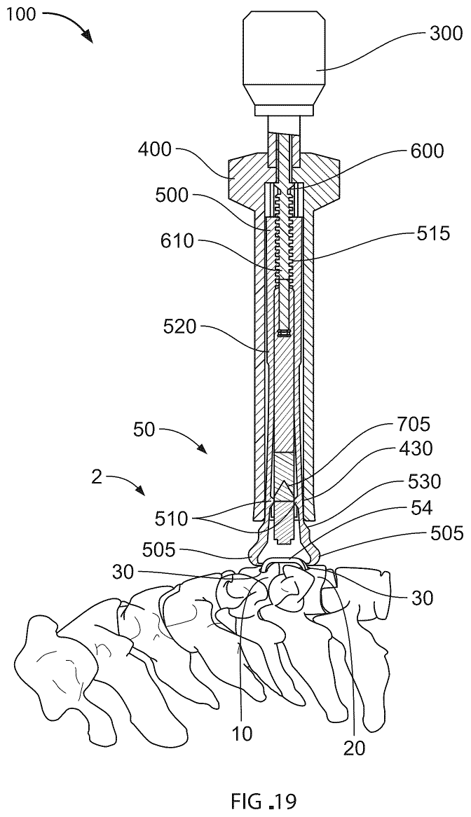

FIG. 19 shows a side partial section view of the inserter assembly 100 as it is disengaged from the implant 50, with the implant 50 in the third orientation 3 within the surgical site (after insertion). Here, a bottom side of the implant bridge 54 may contact the first and/or second bone portions 10, 20. In some embodiments, once the implant 50 has been inserted into the surgical site, the user may turn the knob 300 to cause the locking pin 600 to rotate. As the locking pin 600 rotates, in at least one embodiment, the bender fork threads 515 selectively engage the locking pin threads 610, causing the bender forks 500 to translate within the housing 400. In turn, this may cause the angled portions 530 to engage from the inner surface 430, thereby allowing the prongs 520 to move outwards from each other (e.g., due to stored strain within the bender forks 500). In some embodiments, the prongs 520 may be moved apart by the separating bosses 510 interfacing (or disengaging) with the angled surfaces 705 as the bender forks 500 translate to the first bender fork position. Additionally, the prongs 520 may move outwards from each other such that the hooks 505 disengage from the implant bridge 54. Thus, the inserter assembly 100 may be disengaged from the implant 50.

FIGS. 20 and 21 show an isometric view and an isometric section view of the inserter assembly 100 after inserting the implant 50 into the surgical site, respectively. As shown, once the inserter assembly 100 has been disengaged from the implant 50, the implant legs 52 compress the first bone portion 10 and the second bone portion against each other such that the implant 50 reforms to the third orientation 3. In some cases, after removal from the implant 50, the inserter assembly 100 can be removed by the user.

In certain situations, it may be desirable to remove the implant 50, such as situations of undesirable initial positioning of the implant 50 or fracturing of the implant 50. According to some embodiments, the user may remove the implant 50 from the surgical site by assuming a similar configuration as described with reference to FIG. 18. For instance, the user may position the hooks 505 at the edges 55 of the implant bridge 54. In one or more embodiments, features of the inserter assembly 100 can then be used to move the implant 50 from the third orientation 3 to the second orientation 2 to facilitate easier removal from the surgical site, similar to how the implant 50 is inserted into the surgical site (e.g., with the implant legs 52 in a parallel configuration). Additionally, the retention block 200 may restrain the implant 50 in the first orientation 1 and prevent the inserter assembly 100 from becoming overstressed with the load of storing/grasping the implant 50.

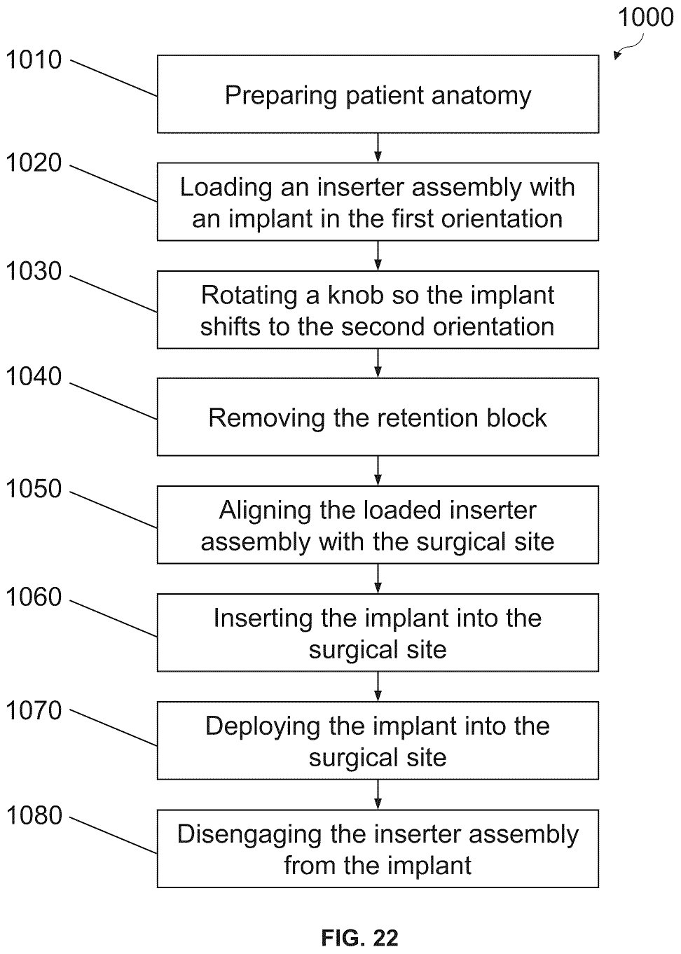

Referring now to FIG. 22, one method 1000 for using the inserter assembly system as described herein generally includes: (1) preparing the patient anatomy 1010; (2) loading the inserter assembly with an implant 1020; (3) rotating the knob of the inserter assembly such that the implant shifts from the first orientation to the second orientation 1030; (4) removing the retention block from the inserter assembly 1040; (5) aligning the loaded inserter assembly with the surgical site 1050; (6) inserting the implant legs into one or more prepared portions of the patient anatomy surrounding the surgical site 1060; (7) deploying the implant into the surgical site 1070 using the inserter assembly; and (8) disengaging the inserter assembly from the implant 1080.

With reference to step 1010, in some embodiments, the user may prepare the patient anatomy by forming an incision to access underlying bones or tissues. In some cases, the user may also form one or more openings within the surgical site via drilling or broaching to prepare the surgical site for implant insertion. For instance, the user may form one or more openings on adjacent bone or tissue sections. Further, the user may verify proper alignment of patient anatomy at the surgical site by resecting or otherwise operating on the patient anatomy to adjust its positioning. In some cases, in preparation for forming an initial incision, the user may scan the patient anatomy via any known imaging techniques (e.g., CT scan, X-rays, etc.) In other cases, the user may scan the patient anatomy at any suitable time while performing the method 1000.

With reference to step 1020, in some embodiments, the inserter assembly may be received pre-loaded with an implant and may be sterilized prior to use by the user. In such embodiments, step 1020 may be optional. In other embodiments, the user may load the inserter assembly with an implant by engaging certain portions of the implant (e.g., the edges of the implant bridge, the implant bridge, the implant legs) with one or more portions of the inserter assembly (e.g., the one or more hooks, the spacer, the retention block, etc.).

With reference to step 1030, in some embodiments, as the user rotates the knob of the inserter assembly in a first direction (e.g., clockwise), the locking pin may rotate and cause the locking pin threads disposed thereon to engage with the bender fork threads disposed on a portion of the bender forks. In turn, this may cause the bender forks to translate from a first bender fork position (e.g., extended) to a second bender fork position (e.g., retracted). At the second bender fork position, the prongs engage with the inner surface of the housing and transmit force to the hooks, applying increased force to the implant as the hooks apply a pulling force to the edges of the implant bridge. In some embodiments, the spacer may be positioned to apply a counter-force to a center portion of the implant bridge (e.g., the bridge) as the hooks apply the pulling force to the edges of the implant bridge. Thus, as the user rotates the knob, the bender forks cause the implant to shift from the first orientation to the second orientation.

With reference to step 1040, in some embodiments, once the implant forms the second orientation, the user may remove the retention block to expose the tips of the implant legs. In some embodiments, step 1040 may be optional.

With reference to step 1050, in some embodiments, the user may align the inserter assembly with the surgical site (e.g., the prepared openings) using imaging techniques, similar to step 1010.

With reference to step 1060, in some embodiments, the user may at least partially insert the tips of the implant legs into prepared portions of the surgical site. For instance, the tips may be inserted into one or more openings formed on adjacent bone or tissue sections. In some cases, the user may perform step 1060 manually (e.g., via pushing or impacting the inserter assembly towards the surgical site), while in other cases, the user may continue rotating the knob to extend the bender forks loaded with the implant towards the surgical site. Additionally, in some cases, the user may perform step 1060 using imaging techniques, similar to step 1010.

With reference to step 1070, in some embodiments, the user may continue rotating the knob in the first direction to continue engaging with the implant. In particular, the bender forks may continue to translate towards the second bender fork position, thereby applying a downwards force to the implant. In this way, the implant legs may be fully inserted within the surgical site such that a bottom portion of the implant bridge engages with the surgical site, thus bridging over the adjacent bone or tissue sections. In certain embodiments, the user may manually push the inserter assembly downwards to fully insert the implant within the surgical site.

With reference to step 1080, in some embodiments, as the user rotates the knob of the inserter assembly in a second direction (e.g., counter-clockwise), the locking pin may rotate and cause the locking pin threads disposed thereon to engage with the bender fork threads disposed on a portion of the bender forks. In turn, this may cause the bender forks to translate from the second bender fork position (e.g., retracted) to the first bender fork position (e.g., extended). At the first bender fork position, the prongs disengage from the inner surface of the housing, thereby reducing the force transmitted to the hooks. Accordingly, the hooks can release the edges of the implant bridge. Thus, as the user rotates the knob in the second direction, the bender forks can deploy the implant into the surgical site, and the implant shifts from the second orientation to the third orientation. In other embodiments, the user may manually pull the inserter assembly upwards to disengage from the deployed implant.

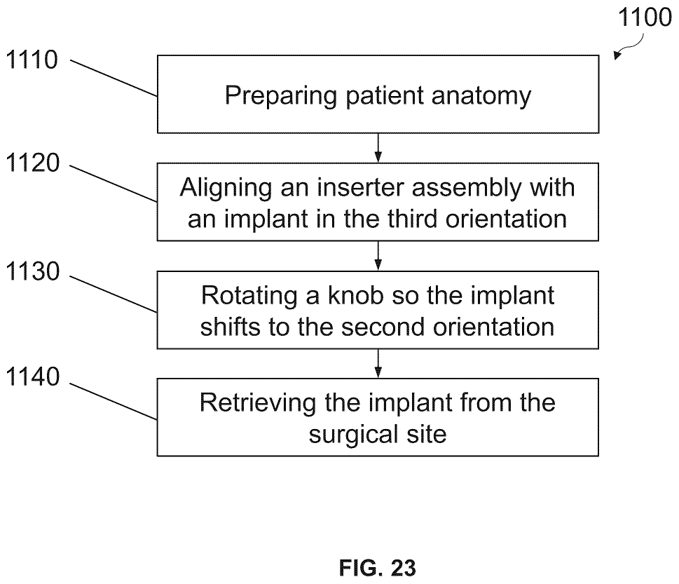

Referring now to FIG. 23, one method 1100 for using an inserter assembly system as described generally includes: (1) preparing the patient anatomy 1110; (2) aligning the inserter assembly with the deployed implant to engage with the implant 1120; (3) rotating the knob of the inserter assembly such that the implant shifts from the third orientation to the second orientation 1130; and (4) retrieving the implant from the surgical site using the inserter assembly 1140.

In some embodiments, step 1110 may be performed similarly to step 1010, wherein the user prepares the underlying patient anatomy with an incision prior to retrieving a deployed implant. In other embodiments, the user may prefer to quickly retrieve an improperly placed or damaged implant shortly after deployment.

In some embodiments, step 1120 may be performed similarly to step 1050, wherein the user may align the inserter assembly with certain portions of the deployed implant (e.g., the edges of the bridge).

In some embodiments, step 1130 may be performed similarly to step 1080. For instance, as the user rotates the knob of the inserter assembly in a first direction (e.g., clockwise), the implant shifts from the third orientation to the second orientation.

In some embodiments, step 1140 may involve the user continuing to rotate the knob in the first direction to continue engaging with the implant. Then, the user may manually pull the inserter assembly upwards to retrieve the implant from the surgical site.

Although the embodiment shown herein is an inserter assembly designed for use with a staple-type fixation implant, the principles of this disclosure may extend to inserter assemblies including any number and positioning of components designed to engage (and disengage) with any type of implants. Various alternate embodiments are contemplated herein, such as, but not limited to, inserter assemblies designed to insert and remove compression implants (e.g., orthopedic screws) or other types of fixation implants (e.g., orthopedic nails) in accordance with the principles of this disclosure.

What is claimed is:

1. A system for inserting or retrieving a surgical implant, the system comprising:
a housing having a first end and a second end, the housing defining a hollow interior compartment, the housing comprising:
an inner surface extending internally within the housing between the first end and the second end of the housing, the inner surface comprising a recess; and
an opening disposed at the first end of the housing, the opening designed to receive a portion of a knob;
a bender fork having a first end and a second end, the bender fork received within and selectively engaging with the recess, the bender fork comprising:
a threaded opening disposed at the first end of the bender fork; and
a prong, the prong further comprising a hook disposed at the second end of the bender fork;
a push rod having a first end and a second end, the push rod positioned adjacent to the bender fork, push rod comprising:
a cavity positioned at the first end of the push rod, the cavity sized to receive a second end of a locking pin;
wherein the locking pin comprises locking pin threads positioned between a first end and the second end of the locking pin, the locking pin received by the threaded opening,
wherein the knob is designed to rotate within the opening and selectively engage with the locking pin, and the locking pin threads selectively engage with the threaded opening, and
wherein the bender fork translates within the recess between at least a first bender fork position and a second bender fork position.

2. The system of claim 1, wherein the push rod further comprises an angled surface that selectively engages the prong.

3. The system of claim 2, wherein the prong further comprises a separating boss that selectively engages the angled surface.

4. The system of claim 3, wherein the prong further comprises an angled portion that selectively engages with the inner surface.

5. The system of claim 1, further comprising a retention block, the retention block receiving a first portion of the surgical implant, the surgical implant forming a first implant orientation.

6. The system of claim 1, further comprising a spacer having a first end, a second end, and at least one distribution wing, the first end of the spacer selectively engaging with the second end of the push rod.

7. The system of claim 6, wherein one or both of the second end and the distribution wing of the spacer selectively engages with the surgical implant.

8. The system of claim 1, wherein the hook selectively engages with a second portion of the surgical implant.

9. An inserter for inserting a surgical implant, the inserter comprising:
a housing having a first end and a second end, the housing defining a hollow interior portion, the interior portion defined by an inner surface extending internally within the housing between the first end and the second end of the housing;
a bender fork having a first end and a second end, the bender fork disposed within the housing, the bender fork comprising:
a first threaded portion positioned at the first end of the bender fork;
at least one prong and at least one hook disposed at the second end of the bender fork, wherein the at least one hook at least partially extends below the second end of the housing;
a separating boss extending from each prong; and
an angled portion selectively engaged with the inner surface;
a push rod disposed within the housing and selectively engaged with the bender fork, the push rod comprising an angled surface selectively engaged with the angled portion of the bender fork;
a locking pin disposed within the housing, the locking pin comprising a second threaded portion rotatably engaged with the first threaded portion, wherein rotation of the locking pin causes the bender fork to translate linearly with respect to the locking pin; and
a knob secured to one end of the locking pin, wherein rotating the knob causes the locking pin to rotate.

10. The inserter of claim 9, further comprising a spacer having a first end and a second end, the first end of the spacer selectively engaging with the second end of the push rod.

11. The inserter of claim 10, wherein the spacer further comprises at least one distribution wing.

12. A method for inserting or retrieving a surgical implant, the method comprising:
identifying a prepared surgical site within a patient;
aligning an inserter assembly with the surgical site, the inserter assembly comprising:
a housing having a first end and a second end and defining an interior compartment;
a knob rotatably attached within the first end of the housing;
a bender fork disposed within the housing, the bender fork comprising at least one prong, the at least one prong further comprising at least one hook extending from the second end of the housing, wherein the bender fork is designed to translate between a first bender fork position and a second bender fork position; and
rotating the knob to translate the bender fork.

13. The method of claim 12, further comprising the step of loading the surgical implant within the inserter assembly, the surgical implant forming a first implant orientation.

14. The method of claim 13, wherein the at least one hook selectively engages with the surgical implant.

15. The method of claim 14, further comprising the step of rotating the knob to translate the bender fork from a first bender fork position to a second bender fork position.

16. The method of claim 15, wherein the at least one prong transmits force to the at least one hook.

17. The method of claim 16, wherein the surgical implant forms a second implant orientation.

18. The method of claim 17, further comprising the step of inserting the surgical implant into the prepared surgical site.

19. The method of claim 18, further comprising the step of rotating the knob to translate the bender fork from the second bender fork position to the first bender fork position.

20. The method of claim 19, further comprising the step of deploying the surgical implant into the prepared surgical site by disengaging the at least one hook from the surgical implant, the surgical implant forming a third implant orientation.
